(19) United States
(12) Patent Application Publication
(10) Pub. No.: US 2006/0120986 A1
(43) Pub. Date: Jun. 8, 2006

(54) Mild cleaning preparation

(76) Inventors: Harald Albrecht, Hamburg (DE); Peter Koch, Hamburg (DE); Stephan Ruppert, Hamburg (DE); Michaela Kohut, Hamburg (DE)

Correspondence Address: GREENBLUM & BERNSTEIN, P.L.C., 1950 ROLAND CLARKE PLACE, RESTON, VA, 20191, US

(21) Appl. No.: 10/534,970

(22) PCT Filed: Jul. 16, 2003
(86) PCT No.: PCT/EP03/07704

§ 371 Date: Dec. 21, 2005

(30) Foreign Application Priority Data
Aug. 7, 2002 (DE) 102 36 063.4

(51) Int. Cl. A61K 8/73; A61K 8/55; A61K 8/34

(52) U.S. Cl. 424/70.13; 424/70.23; 424/70.24

(57) ABSTRACT

Cosmetic preparation containing a) at least two surfactants from the group of the compounds sodium lauryl sarcosinate, sodium monoalkylphosphate, disodium lauryl sulfosuccinate, disodium cocoyl glutamate, α-olefin sulfonate and lauryl glucoside, with a total surfactant concentration of 3 to 1% by weight, b) one or more hydrocolloids in a total concentration of 0.05 to 1% by weight, c) one or more water-soluble salts in a concentration of 1 to 4 % by weight, respectively based on the total weight of the preparation, in addition to optionally further cosmetic or dermatological active ingredients, auxiliary agents and additives and water.

The present invention relates to an aqueous cleansing preparation.

The production of cosmetic cleansing agents has been showing a rising trend for years. This is especially to be attributed to the increasing health consciousness and need for hygiene of consumers.

Cleansing the human body means the removal of (environmental) dirt and thus brings about an increase in psychological and physical well-being. The cleansing of the surface of skin and hair is a very complex operation dependent on many parameters. Firstly, external substances such as, for example, hydrocarbons or inorganic pigments from very diverse surroundings and residues of cosmetics or undesirable microorganisms are to be removed as completely as possible. Secondly, excretions endogenous to the body, such as perspiration, sebum, skin scales and dandruff are to be washed off without serious interventions in the physiological equilibrium.

Cosmetic or dermatological cleansing preparations are so-called “rinse off” preparations which are rinsed off the skin after application. They are generally applied to the parts of the body to be cleansed in the form of a foam with water. Detergent surfactants are the basis of all cosmetic or dermatological cleansing preparations. Surfactants are amphiphilic substances which can dissolve organic, nonpolar substances in water. They are characterized by an ambivalent behavior toward water and lipids: the surfactant molecule contains at least one hydrophilic and one lipophilic group each, which make possible the accumulation on the interface between these two classes of substance. In this way, surfactants provide for a reduction in the surface tension of water, the wetting of the skin, the facilitation of dirt removal and dissolution, easy rinsing and—if desired—also for foam regulation. The basis for dirt removal from lipid-containing soiling is thus afforded.

The hydrophilic moieties of a surfactant molecule are mostly polar functional groups, for example, —COO⁻, —OSO₃²⁻, SO₃⁻, while the hydrophobic moieties are usually nonpolar hydrocarbon radicals. Surfactants are generally classified according to the type and charge of the hydrophilic molecular moiety. In this respect, there are four different groups:
anionic surfactants,
cationic surfactants,
amphoteric surfactants and
nonionic surfactants.

Anionic surfactants generally have carboxylate, sulfate or sulfonate groups as functional groups. In aqueous solution, they form negatively charged organic ions in the acidic or neutral medium. Cationic surfactants are almost exclusively characterized by the presence of a quaternary ammonium group. In aqueous solution, they form positively charged organic ions in the acidic or neutral medium. Amphoteric surfactants contain both anionic and cationic groups and, in aqueous solution, accordingly behave as anionic or cationic surfactants, depending on the pH.

Polyether chains are typical of nonionic surfactants. Nonionic surfactants do not form ions in aqueous medium.

It is understandable that detergent surfactants which are intended to cleanse the skin and hair of fatty and water-soluble dirt constituents also have a degreasing effect on the normal skin lipids. After washing, the skin often feels dry and dull. There has therefore been no lack of attempts to reduce such negative side effects of washing preparations.

One approach to a solution often taken is the use of washing emulsions. In addition to the surfactants, these preparations contain an oil phase, the lipid components of which are intended to (partially) replace the fats removed from the skin during the washing process. However, these products are only effective if they contain a large amount of oils, which considerably increases production costs. Furthermore, with such preparations, the choice of surfactant components is very limited. Since such preparations generally do not lather well, both the cleansing efficiency subjectively perceived by the user and the objective cleansing efficiency of such products is limited. In addition, it is not possible to produce formulations that can be foamed with a pump foamer.

A second approach to a solution is based on preparations that contain conditioners. Skin conditioning agents have the function based on their chemical structure of engaging in polar interactions with the counterstructures of the skin, being absorbed into the skin and remaining there. A perceptible smoothing of the skin thus occurs. One drawback to cleansing preparations containing conditioners is the fact that in some cases conditioners can lead to intolerances, e.g., skin irritations, among particularly sensitive persons. Such usually allergenic reactions can nowadays be proven with respect to virtually all chemical compounds.

There is therefore a need for a plurality of cleansing products with different compositions, so that users suffering from allergies can avoid or select formulas tailored to their allergy profile.

It was therefore the object of the present invention to eliminate or at least reduce the defects of the prior art and to produce easily foamable, aqueous cleansing preparations which produce a pleasant feel on the skin even without skin conditioners.

Surprisingly, the object is attained with a cosmetic preparation containing
a) at least two surfactants from the group of the compounds sodium lauryl sarcosinate, sodium monoalkyl phosphate, disodium lauryl sulfosuccinate, disodium cocoyl glutamate, a-olefin sulfonate, lauryl glucoside with a total surfactant concentration of 3 to 15% by weight,
b) one or more hydrocolloids in a total concentration of 0.05 to 1% by weight,
c) one or more water-soluble salts in a concentration of 1 to 4% by weight, respectively based on the total weight of the preparation,
   in addition to optionally further cosmetic or dermatological active substances, auxiliary agents and additives and water.

The preparations according to the invention are extremely foamable. The foam has a creamy, stable consistency and texture. After the application of the preparation, a pleasant velvety smooth feeling on the skin remains, as is known from preparations containing conditioner.

It is thereby preferred according to the invention if at least two surfactants from the group of the compounds sodium lauryl sarcosinate, sodium monoalkyl phosphate, disodium lauryl sulfosuccinate, disodium cocoyl glutamate, α-olefin sulfonate, lauryl glucoside with a total surfactant concentration of 3 to 15% by weight and particularly preferably in a total concentration of 5 to 12% by weight, respectively based on the total weight of the preparation, are contained in the same.

Furthermore, it is preferred according to the invention to use one or more hydrocolloids in a total concentration of 0.05 to 1% by weight and particularly preferably in a total concentration of 0.1 to 0.5% by weight, respectively based on the total weight of the preparation.

One or more water-soluble salts, preferably in a concentration of 1 to 4% by weight and particularly preferably in a total concentration of 1.5 to 3% by weight, respectively based on the total weight of the preparation, can also be used.

Advantageously, cleansing preparations according to the invention are present in the form of low-viscosity gels and contain one or more gel formers or hydrocolloids.

“Hydrocolloid” is the technical abbreviation for the more correct term “hydrophilic colloid”. Hydrocolloids are macromolecules which have a largely linear structure and have intermolecular forces of interaction, which permit secondary and primary valence bonds between the individual molecules and thus the formation of a reticular structure. Some are water-soluble natural or synthetic polymers which form gels or viscous solutions in aqueous systems. They increase the viscosity of water by either binding water molecules (hydration) or else by absorbing and encapsulating the water into their interwoven macromolecules, at the same time restricting the mobility of the water. Such water-soluble polymers represent a large group of chemically very different natural and synthetic polymers, the common feature of which is their solubility in water or aqueous media. The prerequisite for this is that these polymers have a sufficient number of hydrophilic groups for water solubility and are not too strongly cross-linked. The hydrophilic groups can be non-ionic, anionic or cationic, e.g., as follows:

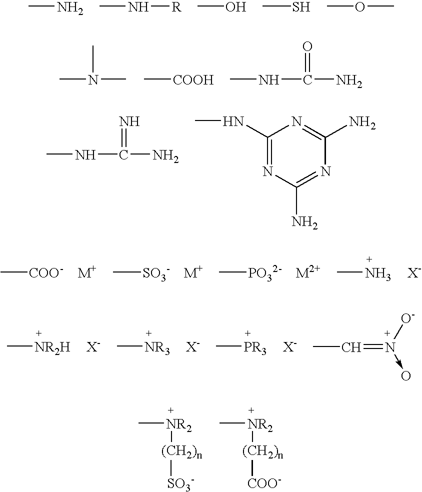

The group of cosmetically and dermatologically relevant hydrocolloids can be divided as follows into:
organic, natural compounds, such as, for example, agar agar, carrageen, tragacanth, gum arabic, alginates, pectins, polyoses, guar gum, carob bean flower, starch, dextrins, gelatins, caseine,
organic, modified natural substances, such as, for example, carboxymethylcellulose and other cellulose ethers, hydroxyethyl- and -propylcellulose and the like,
organic, completely synthetic compounds, such as, for example, polyacrylic and polymethacrylic compounds, vinyl polymers, polycarboxylic acids, polyethers, polyimines, polyamides,
inorganic compounds, such as, for example, polysilicic acids, clay minerals, such as montmorillonites, zeolites, silicas.

Examples of hydrocolloids which are preferred according to the invention are methylcelluloses, which is the name for the methyl ethers of cellulose. They are characterized by the following structural formula 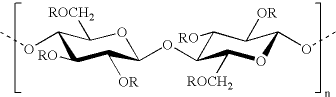
in which R can be a hydrogen or a methyl group.

Particularly advantageous for the purposes of the present invention are the cellulose mixed ethers, which are generally likewise referred to as methylcelluloses, which contain, in addition to a dominating content of methyl groups, also 2-hydroxyethyl, 2-hydroxypropyl or 2-hydroxybutyl groups.

Particular preference is given to (hydroxypropyl)methylcelluloses, for example those available under the trade name Methocel E4M from Dow Chemical Comp.

Also advantageous according to the invention is sodium carboxymethylcellulose, the sodium salt of the glycolic ether of cellulose, for which R in structural formula I can be a hydrogen and/or CH2-COONa. Particular preference is given to the sodium carboxymethylcellulose available under the trade name Natrosol Plus 330 CS from Aqualon and also referred to as cellulose gum.

Also preferred for the purposes of the present invention is xanthan (CAS No. 11138-66-2), also called xanthan gum, which is an anionic heteropolysaccharide which is usually formed by fermentation from corn sugar and is isolated as the potassium salt. It is produced by Xanthomonas campestris and some other species under aerobic conditions and has a molecular weight of from 2×106 to 24×106. Xanthan is formed from a chain having β-1,4-bonded glucose (cellulose) with side chains. The structure of the subgroups comprises glucose, mannose, glucuronic acid, acetate and pyruvate. Xanthan is the name given to the first microbial anionic heteropolysaccharide. It is produced by Xanthomonas campestris and some other species under aerobic conditions and has a molecular weight of from 2-15 106. Xanthan is formed from a chain having β-1,4-bonded glucose (cellulose) with side chains. The structure of the subgroups comprises glucose, mannose, glucuronic acid, acetate and pyruvate. The number of pyruvate units determines the viscosity of the xanthan. Xanthan is produced in two-day batch cultures with a yield of 70-90%, based on carbohydrate used. Yields of 25-30 g/l are achieved thereby. After the culture has been destroyed, work-up takes place by precipitation with, for example, 2-propanol. Xanthan is then dried and ground.

An advantageous gel former for the purposes of the present invention is also carrageen, a gel-forming extract with a similar structure to agar, from North Atlantic red algae, which belong to the Florideae (Chondrus crispus and Gigartina stellata).

The term carrageen is frequently used for the dried algae product and carrageenan for the extract thereof. The carrageen precipitated from the hot-water extract of the algae is a colorless to sand-colored powder with a molecular weight range from 100,000-800,000 and a sulfate content of about 25%. Carrageen, which is very readily soluble in warm water, forms a thixotropic gel upon cooling, even if the water content is 95-98%. The rigidity of the gel is effected by the double helix structure of carrageen. In the case of carrageenan, three main constituents are differentiated: the gel-forming κ fraction consists of D-galactose-4-sulfate and 3,6-anhydro-α-D-galactose, which has alternate glycoside bonds in the 1,3- and 1,4-position (by contrast, agar contains 3,6-anhydro-α-L-galactose). The non-gelling A fraction is composed of 1,3-glycosidically linked D-galactose-2-sulfate and 1,4-bonded D-galactose-2,6-disulfate radicals, and is readily soluble in cold water. The 1-Carrageenan, composed of D-galactose 4-sulfate in 1,3-bond and 3,6-anhydro-α-D-galactose-2-sulfate in 1,4-bond, is both water-soluble and also gel-forming. Further carrageen grades are likewise referred to using Greek letters α, β, γ, μ, v, ξ, π, ω, X. The type of cations present (K+, NH4+, Na+, Mg2+, Ca2+) also influences the solubility of the carrageens.

Polyacrylates are gelling agents likewise to be used advantageously for the purposes of the present invention. Polyacrylates advantageous according to the invention are acrylate-alkyl acrylate copolymers, in particular those chosen from the group of so-called carbomers or carbopols (Carbopol® is actually a registered trademark of NOVEON Inc.). In particular, the acrylate-alkyl acrylate copolymer(s) advantageous according to the invention is/are characterized by the following structure: 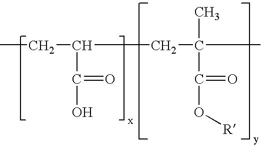
where R′ is a long-chain alkyl radical, and x and y represent numbers which symbolize the respective stoichiometric proportion of each of the comonomers.

According to the invention, preference is given to acrylate copolymers and/or acrylate-alkyl acrylate copolymers which are available under the trade names Carbopol® 1382, Carbopol® 981 and Carbopol® 5984, Aqua SF-1 from NOVEON Inc. and as Aculyn® 33 from International Specialty Products Corp.

Also advantageous are copolymers of C10-30-alkyl acrylates and one or more monomers of acrylic acid, of methacrylic acid or esters thereof which are crosslinked with an allyl ether of sucrose or an allyl ether of pentaerythritol.

Compounds which carry the INCI name “Acrylates/C10-30 Alkyl Acrylate Crosspolymer” are advantageous. Particularly advantageous are those polymers available under the trade names Pemulen TR1 and Pemulen TR2 from NOVEON Inc.

Also advantageous are compounds which the INCI name “acrylates/C12-24 pareth-25 acrylate copolymer” (obtainable under the trade names Synthalen® W2000 from 3V Inc.), which the INCI name “acrylates/steareth-20 methacrylate copolymer” (obtainable under the trade names Aculyn® 22 from International Specialty Products Corp.), which the INCI name “acrylates/steareth-20 itaconate copolymer” (obtainable under the trade names Structure 2001® from National Starch), which the INCI name “acrylates/aminoacrylates/C10-30 alkyl PEG-20 itaconate copolymer” (obtainable under the trade names Structure Plus® from National Starch) and similar polymers.

It is advantageous in terms of the present invention if the content of one or more polyacrylates in the cosmetic or dermatological cleansing preparation is selected from the range of 0.01 to 1% by weight, very particularly advantageous from 0.05 to 0.5% by weight, respectively based on the total weight of the preparations.

According to the invention, the cosmetic preparation according to the invention contains one or more water-soluble salts. These include, e.g., the alkaline and/or alkaline earth halides, carbonates, phosphates, sulfates and/or nitrates.

Advantageously, according to the invention, the salts can be selected from the group sodium chloride, potassium chloride, potassium bromide, calcium chloride, calcium nitrate, calcium iodide, magnesium chloride, barium chloride, lithium chloride, sodium bicarbonate, magnesium sulfate, whereby the salts can also advantageously be used in the form of their hydrates.

According to the invention, the use of the compounds sodium chloride, potassium chloride, ammonium chloride, magnesium sulfate is preferred, with sodium chloride being particularly preferred according to the invention.

The preparation according to the invention can advantageously contain according to the invention one or more preservatives. Advantageous preservatives for the purposes of the present invention are, for example, formaldehyde donors (such as, for example, DMDM hydantoin, which is available, for example, under the trade name Glydant™ from Lonza), iodopropyl butylcarbamates (e.g., those available under the trade names Glycacil-L, Glycacil-S from Lonza and/or Dekaben LMB from Jan Dekker), parabens (i.e., alkyl p-hydroxybenzoates, such as methyl-, ethyl-, propyl- and/or butylparaben), phenoxyethanol, ethanol, benzoic acid and the like. Usually, the preservative system according to the invention further advantageously also comprises preservative assistants, such as, for example, octoxyglycerol, glycine soya, etc. The following table provides an overview of some preservatives that are advantageous according to the invention:

| | |
|---|---|
| E200 | sorbic acid |
| E201 | sodium sorbate |
| E202 | potassium sorbate |
| E203 | calcium sorbate |
| E210 | benzoic acid |
| E211 | sodium benozate |
| E212 | potassium benzoate |
| E213 | calcium benzoate |
| E214 | ethyl p-hydroxy benzoate |
| E215 | ethyl p-hydroxy benzoate |
| | sodium salt |
| E216 | n-propyl p-hydroxy benzoate |
| E217 | n-propyl p-hydroxy benzoate |
| | sodium salt |
| E218 | methyl p-hydroxy benzoate |
| E219 | methyl p-hydroxy benzoate |
| | sodium salt |
| E220 | sulfur dioxide |
| E221 | sodium sulfite |
| E222 | sodium bisulfite |
| E223 | sodium disulfite |
| E224 | potassium disulfite |
| E226 | calcium sulfite |
| E227 | calcium bisulfite |
| E228 | potassium bisulfite |
| E230 | biphenyl (diphenyl) |
| E231 | orthophenylphenol |
| E232 | sodium orthophenylphenolate |
| E233 | thiabendazol |
| E235 | natamycin |
| E236 | formic acid |
| E237 | sodium formate |
| E238 | calcium formate |
| E239 | hexamethylentetramine |
| E249 | potassium nitrite |
| E250 | sodium nitrite |
| E251 | sodium nitrate |
| E252 | potassium nitrate |
| E280 | propionic acid |
| E281 | sodium propionate |
| E282 | calcium propionate |
| E283 | potassium propionate |
| E290 | carbon dioxide |

Preservatives or preservation aids customary in cosmetics are furthermore advantageous, such as dibromodicyanobutane (2-bromo-2-bromomethyl-glutaronitrile), phenoxyethanol, 3-iodo-2-propynylbutyl carbamate, 2-bromo-2-nitro-propane-1,3-diol, imidazolidinylurea, 5-chloro-2-methyl-4-isothiazolin-3-one, 2-chloro-acetamide, benzalkonium chloride, benzyl alcohol.

It is thereby particularly preferred according to the invention if sodium benzoate, sodium salicylate, methyldibromoglutaronitrile and/or phenoxyethanol are used as preservatives.

According to the invention it is advantageous if one more preservatives contains in a total concentration of 0.1 to 1% by weight, preferably 0.15 to 0.8% by weight and very particularly preferably 0.2 to 0.6% by weight, respectively based on the total weight of the preparation.

According to the invention it is advantageous for the preparation according to the invention to contain one or more solubilizers. These can, e.g., support the incorporation of perfume materials. Advantageously according to the invention one or more solubilizers are used in a concentration of 0.1 to 2% by weight, based on the total weight of the preparation. Advantageous solubilizers according to the invention are, e.g., esters of glycerin and fatty acids, in particular hydrogenated fatty acids, etherified with polyethylene and/or polypropylene chains. Thus PEG-100 hydrogenated glyceryl palmitate, PEG-200 hydrogenated glyceryl palmitate and PEG-40 hydrogenated castor oil are preferred according to the invention.

The preparation according to the invention can advantageously contain further surfactants:

Advantageous detergent anionic surfactants for the purposes of the present invention are, e.g.,

acylamino acids and their salts, such as
acylglutamates, in particular sodium acylglutamate
sarcosinates, for example myristoyl sarcosine, TEA lauroyl sarcosinate, sodium lauryl sarcosinate and sodium cocoyl sarcosinate,

sulfonic acids and their salts, such as
acyl isethionates, e.g. sodium/ammonium cocoyl isethionate,
sulfosuccinates, for example dioctyl sodium sulfosuccinate, disodium laureth sulfosuccinate, disodium lauryl sulfosuccinate and disodium undecyleneamido MEA sulfosuccinate

and sulfuric acid esters, such as
alkyl ether sulfate, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

Further
taurates, for example sodium lauroyl taurate and sodium methyl cocoyl taurate,
ether carboxylic acids, for example sodium laureth-13 carboxylate and sodium PEG-6 cocamide carboxylate,
phosphoric acid esters and salts, such as, for example, DEA-oleth-10 phosphate and dilaureth-4 phosphate,
alkylsulfonates, for example sodium coconut monoglyceride sulfate, sodium C₁₂₋₁₄ olefin sulfonate, sodium lauryl sulfoacetate and magnesium PEG-3 cocamide sulfate.
acyl glutamates such as di-TEA-palmitoyl aspartate and sodium caprylic/capric glutamate,
acyl peptides, for example palmitoyl hydrolyzed milk protein, sodium cocoyl hydrolyzed soya protein and sodium/potassium cocoyl hydrolyzed collagen

and carboxylic acids and derivatives, such as
for example lauric acid, aluminum stearate, magnesium alkanolate and zinc undecylenate,
ester carboxylic acids, for example calcium stearoyl lactylate, laureth-6 citrate and sodium PEG-4 lauramide carboxylate,
alkylaryl sulfonates.

Advantageous detergent cationic surfactants for the purposes of the present invention are, e.g., quarternary surfactants. These include
benzalkonium chloride,
alkylbetaine,
alkylamidopropylbetaine
alkylamidopropylhydroxysultaine
alkylamine,
alkylimidazole and
   ethoxylated amines.

Advantageous detergent amphoteric surfactants for the purposes of the present invention are, e.g.,
acyl-/dialkylethylenediamines, for example sodium acyl amphoacetate, disodium acyl amphodipropionate, disodium alkylamphodiacetate, sodium acyl amphohydroxypropylsulfonate, disodium acyl amphodiacetate and sodium acyl amphopropionate,
N-alkylamino acids, e.g., amino propylalkyl glutamide, alkylamino propionic acid, sodium alkylimidodipropionate and lauroamphocarboxy glycinate.

Advantageous detergent nonionic surfactants for the purposes of the present invention are, e.g.,
alkanolamides, such as cocamides MEA/DEA/MIPA,
esters which are formed by esterification of carboxylic acids with ethylene oxide, glycerol, sorbitan or other alcohols,
ethers, for example, ethoxylated alcohols, ethoxylated lanolin, ethoxylated polysiloxanes, propoxylated POE ethers and alkyl polyglycosides, such as lauryl glycoside, decyl glycoside and cocoglycoside,
alcohols.

The preparation according to the invention is advantageously present as an aqueous solution and, in addition to water, can advantageously also contain other constituents, e.g., alcohols, diols or polyols of low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, further alcohols of low C number, e.g., ethanol, isopropanol, 1,2-propanediol and glycerol.

The preparation according to the invention can advantageously contain moisturizing or humectant substances (so-called moisturizers). Advantageous humectant substances for the purposes of the present invention are, e.g., glycerol, lactic acid and/or lactate, in particular sodium lactate, butylene glycol, propylene glycol, biosaccaride gum-1, glycine soy, ethylhexyloxyglycerin, pyrrolidone carboxylic acid and urea. Furthermore, it is in particular advantageous to use polymeric moisturizers from the group of polysaccharides which are water-soluble and/or water-swellable and/or gellable using water. In particular advantageous are, for example, hyaluronic acid, chitosan and/or a fucose-rich polysaccharide which is listed in the Chemical Abstracts under the registry number 178463-23-5 and is available, for example, under the name Fucogel® 1000 from SOLABIA S.A.

It is also advantageous to add complex formers to the preparations according to the invention. The complex formers are advantageously chosen from the group of ethylenediaminetetraacetic acid (EDTA) and anions thereof, nitrilotriacetic acid (NTA) and anions thereof, hydroxyethylenediaminotriacetic acid (HOEDTA) and anions thereof, diethyleneaminopentaacetic acid (DPTA) and anions thereof, trans-1,2-diaminocyclohexanetetraacetic acid (CDTA) and anions thereof, tetrasodium iminodisuccinate, trisodium etylenediamine disuccinate.

An additional content of antioxidants is generally preferred. According to the invention, all the antioxidants suitable or customary for cosmetic and/or dermatological applications can be used as favorable antioxidants.

The preparations according to the invention can also contain all the UVA, UVB and/or broadband filter substances admissible under the Cosmetics Directive.

According to the invention, apart from the substances listed above, the preparations optionally contain the additives customary in cosmetics, for example perfumes, dyes, antimicrobial substances, refatting agents, complexing and sequestering agents, pearlizing agents, further plant extracts, vitamins, active ingredients, bactericides, repellants, self-tanning agents, depigmentation agents, pigments which have a coloring action, emollients, moisturizers and/or humectants, or other customary constituents of a cosmetic or dermatological formulation, such as polymers and foam stabilizers. According to the invention, substances for adjusting the pH value (e.g., citric acid, sodium hydroxide) can also be present in the preparation according to the invention (also in the form of salts thereof).

It is advantageous according to the invention to add glitter substances and/or other effect substances (e.g., incorporated color particles, gas bubbles, active ingredient capsules, color streaks) to the preparation according to the invention.

So-called abrasives (peeling particles), e.g., of small polyethylene beads, can also advantageously be incorporated into the preparation according to the invention.

According to the invention, the preparation according to the invention is advantageously stored in a foam dispenser and is applied from it in the form of a foam. It is advantageous according to the invention if a pump foamer or compressed gas cylinder (also called an aerosol can) is used. It has thereby proven to be particularly advantageous according to the invention if a pump foamer is used as a foam dispenser.

However, the cleansing preparations according to the invention can also advantageously be foamed with a propellant gas. It is thereby advantageous to use the propellant gas in an amount of 0.5 to 30% by weight, particularly preferably in a concentration of 1 to 20% by weight and very particularly preferably in a concentration of 3 to 15% by weight based on the total weight of the formulation.

The propellant gases preferred according to the invention are propane, isobutane and n-butane and mixtures thereof. However, compressed air, carbon dioxide, nitrogen, nitrogen dioxide and dimethyl ether and mixtures of all these gases can also be used advantageously according to the invention. According to the invention, propellant gas mixtures of propane and butane are very particularly preferred.

The person skilled in the art is, of course, aware that there are propellants which are nontoxic per se and in principle suitable for realizing the present invention in the form of aerosol preparations, but which must nevertheless be avoided because of their unacceptable impact on the environment or other accompanying circumstances, in particular fluorinated hydrocarbons and chlorofluorocarbons (CFCs). These gases could also be used advantageously according to the invention.

The use of the cosmetic preparation according to the invention as a cleansing preparation for the skin is advantageous according to the invention.

The use of the preparation according to the invention for cleansing facial skin is in particular in accordance with the invention. With such an application, thereby both cleansing the facial skin of impurities such as, e.g., sebaceous matter, sebum, sweat, dead skin particles, is in accordance with the invention as well as using it to remove decorative cosmetics from the skin (so-called makeup removal). In particular the removal of eye shadow, mascara, foundation, lipstick, powder, etc. is in accordance with the invention.

Furthermore, the products according to the invention are excellently suitable for cleaning objects of everyday life (e.g., dishes, table and cupboard surfaces, cars).

The examples below serve to illustrate the present invention without limiting it. Unless otherwise stated, all amounts, proportions and percentages are based on the weight and the total amount or on the total weight of the preparations.

### EXAMPLES

| | 1 | 2 | 3 | 4 | 5 |
|---|---|---|---|---|---|
| Sodium lauroylsarcosinate | 2.5 | — | — | — | — |
| Sodium monoalkyl phosphate | — | 3.5 | — | 2 | — |
| Disodium lauryl sulfosuccinate | — | — | 5 | — | — |
| Disodium cocoyl glutamate | — | — | — | 2 | 4.5 |
| α-olefin sulfonate | 4 | 4 | — | — | — |
| Lauryl glucoside | — | — | 3 | 2 | 3 |
| Carbomer | 0.1 | — | — | 0.1 | 0.1 |
| Xanthan gum | — | 0.15 | — | — | — |
| Guar gum | — | — | 0.2 | 0.1 | — |
| Sodium chloride | 2.5 | — | — | 1 | — |
| Potassium chloride | — | 3 | — | — | 2 |
| Ammonium chloride | — | — | 2.5 | — | — |
| Magnesium sulfate | — | — | — | 3 | 1 |
| PEG-200 hydrogenated glyceryl | 0.5 | — | — | — | — |
| palmitate | | | | | |
| PEG-40 hydrogenated castor oil | 0.1 | 0.1 | 0.1 | 0.1 | 0.1 |
| PEG-100 hydrogenated glyceryl | — | 0.5 | — | — | 0.5 |
| palmitate | | | | | |
| Sodium benzoate | 0.5 | 0.5 | — | 0.5 | 0.5 |
| Sodium salicylate | — | 0.2 | — | 0.2 | 0.2 |
| Methyldibromoglutaronitrile | — | — | 0.04 | — | — |
| Phenoxyethanol | — | — | 0.16 | — | — |
| Citric acid | q.s. | q.s. | q.s. | q.s. | q.s. |
| Perfume | q.s. | q.s. | q.s. | q.s. | q.s. |
| Water | ad | ad | ad | ad | ad |
| | 100 | 100 | 100 | 100 | 100 |

1. Cosmetic preparation composition containing
a) at- least two surfactants from sodium lauryl sarcosinate, sodium monoalkyl phosphate, disodium lauryl sulfosuccinate, disodium cocoyl glutamate, α-olefin sulfonate and lauryl glucoside with a total surfactant concentration of 3 to 15% by weight;
b) at least one hydrocolloid in a total concentration of 0.05 to 1% by weight; and
c) at least one water-soluble salt in a concentration of 1 to 4% by weight,
respectively based on the total weight of the preparation composition.

2. Cosmetic preparation composition according to claim 1, wherein the at least one hydrocolloid comprises at least one compound of agar agar, carrageen, tragacanth, gum arabic, alginates, pectins, polyoses, guar gum, carob bean flower, starch, dextrins, gelatins, caseine, carboxymethylcellulose and other cellulose ethers, hydroxyethyl- and -propylcellulose, polyacrylic and polymethacrylic compounds, vinyl polymers, polycarboxylic acids, polyethers, polyimines, polyamides polysilicic acids, clay minerals, zeolites, and silicas.

3. Cosmetic preparation composition according to claim 1, wherein the at least one water-soluble salt comprises at least one of sodium chloride, potassium chloride, ammonium chloride, and magnesium sulfate.

4. Cosmetic preparation composition according to claim 1, including at least one preservative in a total concentration of 0.1 to 1% by weight, based on the total weight of the preparation composition.

5. Cosmetic preparation composition according to claim 1, including at least one solubilizer in a concentration of 0.1 to 1% by weight based on the total weight of the preparation composition.

6. Cosmetic preparation composition according to claim 1, wherein the preparation composition is stored as a foamable preparation in a pump foamer or an aerosol can and can be applied from the pump foamer or the aerosol can.

7. Method of cleansing skin comprising applying a cosmetic preparation composition according to claim 1 to the skin.

8. Cosmetic preparation composition according to claim 1, including water.

9. Cosmetic preparation composition according to claim 1, including cosmetic or dermatological active substances, auxiliary agents and additives.

10. Cosmetic preparation composition according to claim 2, wherein the at least one water-soluble salt comprises at least one of sodium chloride, potassium chloride, ammonium chloride, and magnesium sulfate.

11. Cosmetic preparation composition according to claim 8, wherein the at least one water-soluble salt comprises at least one of sodium chloride, potassium chloride, ammonium chloride, and magnesium sulfate.

12. Cosmetic preparation composition according to claim 2, including at least one preservative in a total concentration of 0.1 to 1% by weight, based on the total weight of the preparation composition.

13. Cosmetic preparation composition according to claim 8, including at least one preservative in a total concentration of 0.1 to 1% by weight, based on the total weight of the preparation composition.

14. Cosmetic preparation composition according to claim 2, including at least one solubilizer in a concentration of 0.1 to 1% by weight based on the total weight of the preparation composition.

15. Cosmetic preparation composition according to claim 4, including at least one solubilizer in a concentration of 0.1 to 1% by weight based on the total weight of the preparation composition.

16. Cosmetic preparation composition according to claim 8, including at least one solubilizer in a concentration of 0.1 to 1% by weight based on the total weight of the preparation composition.

17. Cosmetic preparation composition according to claim 2, wherein the preparation composition is stored as a foamable preparation in a pump foamer or an aerosol can and can be applied from the pump foamer or the aerosol can.

18. Cosmetic preparation composition according to claim 8, wherein the preparation composition is stored as a foamable preparation in a pump foamer or an aerosol can and can be applied from the pump foamer or the aerosol can.

19. Cosmetic preparation composition according to claim 2, wherein the clay minerals comprise montmorillonites.

20. Method of cleansing skin comprising applying a cosmetic preparation composition according to claim 8 to the skin.
